(19) United States
(12) Patent Application Publication
(10) Pub. No.: US 2008/0113021 A1
(43) Pub. Date: May 15, 2008

(54) IBUPROFEN COMPOSITION

(76) Inventor: Robert Shen, North Wales, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/877,917

(22) Filed: Oct. 24, 2007

Related U.S. Application Data
(60) Provisional application No. 60/854,349, filed on Oct. 25, 2006.

(51) Int. Cl. A61K 31/60; A61K 31/192; A61K 9/20

(52) U.S. Cl. 424/464; 514/165; 514/570; 514/569; 514/290

(57) ABSTRACT

The invention relates to an immediate release tablet capable of being chewed or disintegrated in the oral cavity, which comprises a plurality of particles in a matrix. The particles contain an active ingredient, such as ibuprofen, and a first water soluble acid having a solubility greater than about 10 g/100 mL water at 20° C.; and the matrix contains a second water soluble acid having a solubility less than about 5 g/100 mL water at 20° C.

The present invention relates to ibuprofen compositions, more specifically to ibuprofen compositions with reduced throat burn characteristics.

### BACKGROUND OF THE INVENTION

Many flavors and sweeteners have been added to medications in order to make them more palatable and to mask the unpleasant taste and aftertaste that is commonly associated with such products. Certain medicinal ingredients, in addition to having an unpleasant taste, create a burning or scratching sensation in the throat when swallowed. This can be expressed through a throat catch or cough. Unfortunately, flavors and sweeteners do little to overcome this throat burning sensation. Despite numerous efforts to find an effective means to eliminate this burn, there is a continuing need for a method to effectively eliminate the burning sensation with medications, or at least to reduce the burn to a level such that a chewable composition can be provided.

Ibuprofen is a well known medication which possesses an unpalatable burning sensation in the mouth and throat after ingestion. Similarly, ketoprofen also often possesses such sensations.

Several approaches for overcoming this burning sensation have been proposed in the art. U.S. Pat. No. 6,627,214 discloses a method for inhibiting the burn sensation of racemic mixtures of propionic acid derivatives by generally providing fumaric acid in an amount, relative to the propionic acid derivative dosage, of about 50 to about 150 weight percent. While fumaric acid can be effective at lowering the burn sensation, proportionally higher levels of fumaric acid may contribute to a level of sourness, which could render convenience dosage forms such as fast dissolving and chewable tablets less palatable. Another approach is to coat the ibuprofen particles with a hydro-colloid and fumaric acid in order to minimize the irritation to the mucous membranes of the throat as disclosed in U.S. Pat. No. 4,762,702. Because of their hydrophilicity, hydro-colloids permit water to be quickly absorbed into the drug particle upon ingestion, which disadvantageously reduces the burn masking effect of the coating. Yet a further approach is to mix an acid compound, such as fumaric acid, with an active ingredient coated with a tastemasking membrane comprising polymers that are insoluble in an acidic environment and soluble at pH 5 or higher as disclosed in U.S. Pat. No. 5,409,711.

It would be desirable to have a chewable or disintegrable, immediate release dosage form that would not possess the burn typically associated with some active ingredients, such as ibuprofen.

### SUMMARY OF THE INVENTION

This invention relates to dosage forms capable of being chewed or disintegrated in the oral cavity prior to swallowing, comprising, consisting of, and/or consisting essentially of

a. a plurality of particles comprising, consisting of, and/or consisting essentially of (i) a propionic acid derivative, such as ibuprofen, and (ii) a taste-masking effective amount of a water soluble acid having a solubility greater than about 10 g/100 mL water at 20° C.; and

b. a matrix comprising, consisting of, and/or consisting essentially of an acid having a solubility less than about 5 g/100 mL water at 20° C. as claimed herein.

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. As used herein, all percentages are by weight unless otherwise specified. In addition, all ranges set forth herein are meant to include any combinations of values between the two endpoints, inclusively.

As used herein, the term “immediate release” shall mean that the dissolution of the dosage form conforms to USP specifications for immediate release tablets containing the particular active ingredient employed. For example, for acetaminophen tablets, USP 24 specifies that in pH 5.8 phosphate buffer, using USP apparatus 2 (paddles) at 50 rpm, at least 80% of the acetaminophen contained in the dosage form is released therefrom within 30 minutes after dosing, and for ibuprofen tablets, USP 24 specifies that in pH 7.2 phosphate buffer, using USP apparatus 2 (paddles) at 50 rpm, at least 80% of the ibuprofen contained in the dosage form is released therefrom within 60 minutes after dosing. See USP 24, 2000 Version, 19-20 and 856 (1999).

The term, “good mouth feel” shall mean that the dosage form becomes a slippery, gel-like mass capable of suspending gritty particles during mastication. By “high weight average molecular weight” it is meant a weight average molecular weight between about 500,000 to about 10,000,000, e.g. from about 1,000,000 to about 7,000,000.

The term, “burn” is understood to mean the commonly identified peppery or irritating sensation in the throat and/or mouth, often noted when taking ibuprofen and related compounds. This burn is different than bitterness inasmuch as the addition of a sweetener is not effective in reducing the sensation. The burn can be expressed as a throat catch, or as a cough that results from the irritation.

As used herein, a “high solubility acid” shall mean an acid having a solubility greater than 10.0 g/100 mL, e.g., greater than about 60 g/100 mL water at 20° C.

As used herein, a “low solubility acid” shall mean an acid having a solubility less than 5.0 g/100 mL, e.g. less than 0.63 g/100 mL water at 20° C.

As used herein, a “tastemasking effective amount” shall mean the amount of a component that is necessary to tastemask the propionic acid derivative contained in the dosage form. Although this amount may vary based upon, for example, the type and amount of propionic acid derivative selected, typically this amount may range from about, based upon the total weight of the dosage form, from about 1% to about 40%.

“Enteric” shall mean being able to be dissolved at a pH greater than that of the stomach, i.e., e.g., at a pH of greater than about 5.0 or greater than about 5.5 or greater than about 6.0 or that which is found in the intestines.

As used herein, the term “dosage form” applies to any ingestible forms, including confections. In one embodiment, dosage forms are solid, semi-solid, or liquid compositions designed to contain a specific pre-determined amount of a certain ingredient, for example an active ingredient as defined below. Suitable dosage forms may be pharmaceutical drug delivery systems, including those for oral administration, buccal administration, and the like. In one embodiment, the dosage forms of the present invention are considered to be solid; however, they may contain liquid or semi-solid components. In another embodiment, the dosage form is an orally administered system for delivering a pharmaceutical active ingredient to the gastrointestinal tract of a human. In yet another embodiment, the dosage form is an orally administered “placebo” system containing pharmaceutically inactive ingredients, and the dosage form is designed to have the same appearance as a particular pharmaceutically active dosage form, such as may be used for control purposes in clinical studies to test, for example, the safety and efficacy of a particular pharmaceutically active ingredient. In one embodiment, the dosage form contains all active ingredients within the same solid, semi-solid, or liquid forms. In another embodiment, the dosage form contains the active ingredients in one or more solid, semi-solid, or liquid forms. In one embodiment, the dosage form is a chewable tablet that is beneficial to those who have difficulty in swallowing a tablet.

The dosage form of the present invention is made from a composition comprising (a) a plurality of particles comprised of a propionic acid derivative; a tastemasking effective amount of a high solubility acid; and optionally one or more secondary active ingredients; and (b) a matrix comprised of a low solubility acid. The particles may optionally be coated with a polymeric coating layer.

In one embodiment, the dosage form is comprised of, based upon the total weight of the dosage form, from about 1 percent to about 50 percent, e.g., from about 1 percent to about 25 percent of the coated particles and from about 50 percent to about 99 percent, e.g., from about 75 percent to about 95 percent of the matrix.

The core of the coated particles are comprised of, based upon the total dry weight of the coated particles, from about 5 percent to about 90 percent, e.g., from about 25 percent to about 80 percent of a propionic acid derivative; from about 0 percent to about 50 percent, e.g., from about 0.1 percent to about 25 percent of an optional secondary active ingredient; and from about 1 percent to about 20 percent, e.g., from about percent to about 15 percent of a high solubility acid.

The coated particles are comprised of, based upon the total dry weight of the coated particles, from about 50 percent to about 95 percent, i.e., e.g., from about 70 percent to about 90 percent of the granulation core and from about 5 percent to about 50 percent, i.e., e.g., about 10 percent to about 30 percent of a polymeric coating layer.

In addition to optional excipients commonly used in dosage forms, the matrix is also comprised of, based upon the total weight of the matrix, from about 0.1 percent to about 30 percent, e.g., from about 0.5 percent to about 20 percent or from about 1 percent to about 10 percent of a low solubility acid.

The weight ratio of high solubility acid in the particle to low solubility acid in the matrix is from about 1 part to about 50 parts:about 99 parts to about 50 parts, i.e., e.g., from about 1 part to about 10 parts:about 99 parts to about 90 parts.

Propionic acid derivatives are a well known class of pharmaceutically acceptable analgesics/non-steroidal anti-inflammatory drugs, which typically have a free —CH(CH₃)COOH or —CH₂CH₂COOH or a pharmaceutically acceptable salt group, such as —CH(CH 3) COO—Na+ or CH₂CH₂COO—Na+, which are typically attached directly or via a carbonyl functionality to an aromatic ring system. Examples of suitable propionic acid derivatives include, but are not limited to, ibuprofen, naproxen, benoxaprofen, naproxen sodium, flurbiprofen, fenoprofen, fenbuprofen, ketoprofen, indoprofen, pirprofen, carpofen, oxaprofen, pranoprofen, microprofen, tioxaprofen, suproprofen, alminoprofen, tiaprofenic acid, fluprofen and bucloxic acid. The structural formula is exemplified in U.S. Pat. No. 4,923,898. Propionic acid derivatives are typically administered on a daily basis, with the daily dose ranging from about 50 milligrams to about 2000 milligrams, e.g., from about 100 milligrams to 1600 milligrams or from about 200 milligrams to about 1200 milligrams.

Ibuprofen is a widely used, well known non-steroidal anti-inflammatory propionic acid derivative. Ibuprofen is chemically known as 2-(4-isobutylphenyl)-propionic acid. As used herein ibuprofen is understood to include 2-(4-isobutylphenyl)propionic acid as well as the pharmaceutically acceptable salts. Suitable ibuprofen salts include, but are not limited to arginine, lysine, histidine, as well as other salts described in U.S. Pat. Nos. 4,279,926, 4,873,231, 5,424,075 and 5,510,385. Other examples of suitable pharmaceutically acceptable salts of ibuprofen include ibuprofen lysinate, dexibuprofen lysinate, the sodium salt of ibuprofen; and racemic and individual purified forms of S(+)-ibuprofen and R(−)-ibuprofen enantiomers.

High solubility acids suitable for use in the particles include, but are not limited to certain amino acids such as alanine, arginine, glucine, proline, lycine, threonine; glutaric acid, ascorbic acid, malic acid, oxalic acid, tartaric acid, malonic acid, acetic acid, citric acid and mixtures thereof.

Suitable secondary active ingredients, which may be included within the particles and/or the matrix, include other pharmaceuticals, minerals, vitamins, other nutraceuticals, and mixtures thereof. Suitable pharmaceuticals include analgesics, anti-inflammatory agents, antiarthritics, anesthetics, antihistamines, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, diuretics, expectorants, gastrointestinal agents, migraine preparations, motion sickness products, mucolytics, muscle relaxants, osteoporosis preparations, polydimethylsiloxanes, respiratory agents, sleep aids, urinary tract agents and mixtures thereof.

Examples of suitable gastrointestinal agents include stimulant laxatives, such as bisacodyl, cascara sagrada, danthron, senna, phenolphthalein, aloe, castor oil, ricinoleic acid, and dehydrocholic acid, and mixtures thereof; H2 receptor antagonists, such as famotidine, ranitidine, cimetadine; proton pump inhibitors; gastrointestinal cytoprotectives, such as sucraflate and misoprostol; gastrointestinal prokinetics, such as Prucalopride, antibiotics for H. pylori, such as clarithromycin, amoxicillin, tetracycline, and metronidazole; antidiarrheals, such as diphenoxylate and loperamide; glycopyrrolate; antiemetics, such as ondansetron, analgesics, such as mesalamine.

In one embodiment, the secondary active agent may be selected from bisacodyl, famotidine, ranitidine, cimetidine, prucalopride, diphenoxylate, loperamide, lactase, mesalamine, bismuth, antacids, and pharmaceutically acceptable salts, esters, isomers, and mixtures thereof.

In another embodiment, the secondary active agent may be selected from acetaminophen; acetyl salicylic acid; diclofenac; cyclobenzaprine; meloxicam; cox-2 inhibitors such as rofecoxib and celecoxib; codeine; oxycodone; hydrocodone; tramadol; and pharmaceutically acceptable salts, esters, isomers, and mixtures thereof.

In another embodiment, the active agent may be selected from pseudoephedrine; phenylepherine; methocarbamol; doxylamine; guaifenesin; antacids; simethicone; cyclobenzaprine; chloroxazone; glucosamine; chondroitin; phenylpropanolamine; chlorpheniramine; dextromethorphan; diphenhydramine; astemizole; terfenadine; fexofenadine; loratadine; cetirizine; mixtures thereof and pharmaceutically acceptable salts, esters, isomers, and mixtures thereof.

Examples of suitable polydimethylsiloxanes, which include, but are not limited to dimethicone and simethicone, are those disclosed in U.S. Pat. Nos. 4,906,478, 5,275,822, and 6,103,260, the contents of each is expressly incorporated herein by reference. As used herein, the term “simethicone” refers to the broader class of polydimethylsiloxanes, including but not limited to simethicone and dimethicone.

The secondary active ingredient(s) are present in the dosage form in a therapeutically effective amount, which is an amount that produces the desired therapeutic response upon oral administration and can be readily determined by one skilled in the art. In determining such amounts, it is well known in the art that various factors must be considered that include, but are not limited to the particular active ingredient being administered, the bioavailability characteristics of the active ingredient, the dose regime, and the age and weight of the patient.

In one embodiment the particles are produced in two steps, including an initial granulation step (i.e. to yield granulated particles) and a secondary coating step, in which the granulated particles from the first step are coated with a polymer coating (i.e. to yield coated particles). The average particle size of the uncoated granulated particles may vary, but typically range between from about 20 microns to about 800 microns, e.g. from about 50 microns to about 600 microns, or from about 100 microns to about 400 microns.

In one embodiment, the core granulation may contain, based upon the total dry weight of the coated particles, from about 10 percent to about 50 percent of dextrose monohydrate, e.g. from about 20 percent to about 50 percent of dextrose monohydrate. In another embodiment, the core granulation may contain, based upon the total dry weight of the coated particles, from about 1 percent to about 10 percent hypromellose, e.g. from about 1 percent to about 5 percent of hypromellose.

In embodiments wherein the particles are optionally coated, the coating layer may be a taste-masking polymeric coating layer. In one embodiment, the coating layer is comprised of any enteric polymer known in the art. Suitable enteric polymers include, but are not limited to, hydroxypropyl methylcellulose phthalate, hydroxypropyl methylcellulose acetate succinate, shellac, cellulose acetate phthalate polyvinylacetate phthalate, polymethacrylate-based polymers, and copolymers and mixtures thereof. Examples of suitable polymethacrylate-based polymers include, but are not limited to poly(methacrylic acid, methyl methacrylate) 1:2, which is commercially available from Rohm Pharma GmbH under the tradename, “EUDRAGIT S” polymers, and poly(methacrylic acid, methyl methacrylate) 1:1, which is commercially available from Rohm Pharma GmbH under the tradename, “EUDRAGIT L” polymers. In one embodiment, the enteric polymer is selected from non-acrylate compounds, such as hydroxypropyl methylcellulose phthalate, hydroxypropyl methylcellulose acetate succinate, cellulose acetate phthalate, polyvinylacetate phthalate, and copolymers and mixtures thereof.

In another embodiment, the enteric polymer may be mixed with a film forming, water insoluble polymer at a ratio of about 90:10 to about 10:90. Suitable film forming, water insoluble polymers include, but are not limited to, polyvinyl acetate, cellulose acetate, ethylcellulose, cellulose acetate butyrate, and mixtures thereof.

In another embodiment, the enteric polymer may be mixed with a film forming, water soluble polymer at a ratio of about 95:5 to about 70:30. Examples of suitable film forming, water soluble polymers include, but are not limited to, polyvinylalcohol (PVA), hydroxypropyl starch, hydroxyethyl starch, pullulan, methylethyl starch, carboxymethyl starch, methylcellulose, hydroxypropylcellulose (HPC), hydroxyethylmethylcellulose (HEMC), hydroxypropylmethylcellulose (HPMC), hydroxybutylmethylcellu lose (HBMC), carboxymethylcellu lose (CMC), hydroxyethylethylcellulose (HEEC), hydroxyethylhydroxypropylmethyl cellulose (HEMPMC), starches, and polymers and derivatives and mixtures thereof.

In another embodiment, the optional coating layer on the particles may be comprised of a mixture of film forming, water insoluble polymers and film forming, water soluble polymers at a ratio of about 99:1 to about 70:30.

Optionally, the coating layer may also include, based on the total dry weight of the coated particle, from about 0.1 percent to about 15 percent of a plasticizer, i.e., e.g. from about 0.2 percent to about 10 percent of a plasticizer. Examples of suitable plasticizers include, but are not limited to, polyethylene glycol; propylene glycol; glyceryl monostearate; glycerin; sorbitol; triethyl citrate; tributyl citrate; dibutyl sebecate; vegetable oils such as castor oil, rape oil, olive oil, and sesame oil; surfactants such as polysorbates, sodium lauryl sulfates, and dioctyl-sodium sulfosuccinates; mono acetate of glycerol; diacetate of glycerol; triacetate of glycerol; natural gums; triacetin; acetyltributyl citrate; diethyloxalate; diethylmalate; diethyl fumarate; diethylmalonate; dioctylphthalate; dibutylsuccinate; glyceroltributyrate; glycerol monostearate; hydrogenated castor oil; substituted triglycerides and glycerides; and the like and/or mixtures thereof. In one embodiment the plasticizer comprises a blend of glyceryl monostearate and triethyl citrate.

In another embodiment, the coating layer of the coated particle contains, based upon the total dry weight of the coating layer, from about 1 percent to about 25 percent of a plasticizer, e.g. from about 1 percent to about 20 percent of a plasticizer; and from about 75 percent to about 99 percent of an enteric polymer, e.g. from about 80 percent to about 99 percent of an enteric polymer. The coating layer can also include, based upon the total dry weight of the coated particles, from about 0.1 percent to about 25 percent of the optional secondary active ingredient(s).

The average particle size of the coated particles also may vary, but typically will range between from about 40 microns to about 1000 microns, e.g., from about 100 microns to about 700 microns or from about 150 microns to about 500 microns.

Optional ingredients for use in the granulation core of the coated particle include binders, fillers, glidants, flavors, disintegrants, lubricants, sweeteners, sensates, and mixtures thereof. Examples of suitable binders include, but are not limited to hypromellose, hydroxypropyl cellulose, methylcellulose, microcrystalline cellulose and starch.

In one embodiment, the particle utilizes starch or a starch derivative as a binder. As used herein, “modified starches” include starches that have been modified by crosslinking, chemically modified for improved stability, or physically modified for improved solubility properties. As used herein, “pre-gelatinized starches” or “instantized starches” refers to modified starches that have been pre-wetted, then dried to enhance their cold-water solubility. Suitable modified starches are commercially available from several suppliers such as, for example, A.E. Staley Manufacturing Company, and National Starch & Chemical Company. One suitable modified starch includes the pre-gelatinized waxy maize derivative starches that are commercially available from National Starch & Chemical Company under the tradenames, “Purity Gum” and “FilmSet”, and derivatives, copolymers, and mixtures thereof. Such waxy maize starches typically contain, based upon the total weight of the starch, from about 0 percent to about 18 percent of amylose and from about 100 percent to about 88 percent of amylopectin.

Examples of suitable fillers include, but are not limited to dextrose monohydrate, mannitol, lactitol, maltodextrin, sucrose, fructose, lactose, lactose monohydrate and the like, and mixtures thereof. Examples of suitable sweeteners include, but are not limited to aspartame, acesulfame potassium, neotame, sucralose, saccharine, and associated salts thereof, and mixtures thereof. Examples of suitable disintegrants include, but are not limited to cross linked povidone, sodium starch glycolate, cross-carmellose sodium, and mixtures thereof. Examples of suitable lubricants include, but are not limited to stearic acid, magnesium stearate, and mixtures thereof. The granulation core mixture may also incorporate pharmaceutically acceptable adjuvants, including, for example, preservatives; flavors such as, for example, orange and/or vanilla; acidulants; glidants; surfactants; and coloring agents such as, for example, FD&C yellow.

Examples of suitable sensates, which may be included in the granulation core and/or the coating layer of the particle, include, but are not limited to, cooling or warming compounds. Suitable non-volatile cooling agents include, but are not limited to menthyl esters, carboxamides, ureas, phosphine oxides, and mixtures thereof. In one embodiment, such sensates are used in an amount such that the agents are substantially free from odor or odorless vapor and thus do not lose more than about 1% by weight when placed in an open container at 50° C. for at least one hour. Typically such agents may have an average molecular weight of greater than 300 atomic molecular units (amu) or more. One example of such a non-volatile cooling agents is the menthyl ester mixture commercially available from International Flavors & Fragrances under the tradename, “Cooler #2”. Other cooling agents for use in the particle include wintergreen, menthol, spearmint, menthol derivatives, and mixtures thereof.

Low solubility acids suitable for use in the matrix include, but are not limited to oleic acid, stearic acid, certain amino acids such as aspartic acid, glutamic acid, glutamine, histidine, isoleucine, leucine, methionone, phenylalanine, serine, tryptophan, tyrosine, valine, and fumaric acid, and mixtures thereof. The concentration of low solubility acids present to inhibit the burn of propionic acid derivative will vary on the amount of burn reduction desired. Generally the level of the low solubility acids is from about 1 percent to about 40 percent, e.g., from about 5 percent to about 35 percent or from about 10 percent to about 30 percent of the propionic acid derivative amount or dosage. Typically the level of low solubility acid is, based upon the weight of the final dosage form, from about 0.1 percent to about 20 percent, e.g., from about 0.1 percent to about 6 percent.

The matrix may optionally contain other conventional auxiliary ingredients, such as fillers; conventional dry binders including but not limited to microcrystalline cellulose, dextrose monohydrate, and the like; sweeteners; disintegrants; and lubricants such as, for example, stearic acid, magnesium stearate, and mixtures thereof. The mixture may also incorporate pharmaceutically acceptable adjuvants, including, for example, preservatives; flavors such as, for example, orange and/or vanilla; acidulants; glidants; surfactants; and coloring agents such as, for example, FD&C yellow. In one embodiment, the matrix comprises no more than about 25 weight % of such optional auxiliary ingredients.

In one embodiment, the matrix may also or either incorporate any of the aforementioned sensates.

The dosage form may be made in any manner, and for tablet dosage forms, a variety of tableting methods are known in the art. Conventional methods for tablet production include direct compression (“dry blending”), dry granulation followed by compression, and wet granulation followed by drying and compression. Other methods include the use of compacting roller technology such as a chilsonator or drop roller, or molding, casting, or extrusion technologies. All of these methods are well known in the art, and are described in detail in, for example, Lachman, et al., The Theory and Practice of Industrial Pharmacy, Chapter 11, (3^{rd} Ed. 1986), which is incorporated by reference herein.

In the direct compression tableting method, a blend of the propionic acid derivative, low solubility acid, optional secondary active ingredient and any other appropriate optional ingredients are granulated, then optionally coated with an enteric polymer coating. The particles are then directly compacted with the high solubility acid and other appropriate matrix ingredients. After all ingredients are blended together, a pre-determined volume of particles from the blend is filled into a die cavity of a rotary tablet press, which continuously rotates as part of a “die table” from the filling position to a compaction position. The particles are compacted between an upper punch and a lower punch to an ejection position, at which the resulting tablet is pushed from the die cavity by the lower punch and guided to an ejection chute by a stationary “take-off” bar. Advantageously, the direct compression method minimizes or eliminates the use of water-soluble, non-saccharide polymeric binders such as polyvinyl pyrrolidone, alginates, hydroxypropyl cellulose, hydroxypropylmethylcellulose, hydroxyethylcellulose, and the like, which can adversely effect dissolution.

In one embodiment, the tableting method is carried out such that the resulting tablet is relatively soft. The hardness of a “soft” tablet produced in accordance with the present invention is up to about 15 kiloponds per square centimeter (kp/cm²), i.e., e.g., from about 1 kp/cm² to 8 kp/cm² or from about 2 kp/cm² to 6 kp/cm². Hardness is a term used in the art to describe the diametrical breaking strength as measured by conventional pharmaceutical hardness testing equipment, such as a Schleuniger Hardness Tester. In order to compare values across differently-sized tablets, the breaking strength is normalized for the area of the break (which may be approximated as tablet diameter times thickness). This normalized value, expressed in kp/cm², is sometimes referred in the art as “tablet tensile strength.” A general discussion of tablet hardness testing is found in Leiberman et al., Pharmaceutical Dosage Forms—Tablets, Volume 2, 2^{nd} ed., Marcel Dekker Inc., 1990, pp. 213-217, 327-329, which is incorporated by reference herein.

We have unexpectedly found that the addition of high solubility acid to the propionic acid derivative-containing particle and a low solubility acid to the tablet matrix results in a dosage form that not only delivers a good mouthfeel, but also surprisingly does so without a significant throat burning sensation.

Specific embodiments of the present invention are illustrated by way of the following examples. This invention is not confined to the specific limitations set forth in these examples, but rather to the scope of the appended claims. Unless otherwise stated, the percentages and ratios given below are by weight.

### EXAMPLES

### Example 1

### Ibuprofen Tablets Formula A

### Part A: Preparation of Ibuprofen Granulation Formula A

A granulation comprised of the ingredients set forth in Table A below was made by initially combining ibuprofen, dextrose, citric acid, and hypromellose with mixing at an air flow rate of 3.6 scfm in a Glatt 5/9 top spray fluid bed granulator to form a granulation mixture.

In a laboratory mixer set at 75 RPM, 90 g of starch was added to 1497 g of cold water with mixing to produce a starch paste solution having 5.67% solids. This solution was heated while mixing until it reached 75° C. The resulting solution was then sprayed into the granulation mixture at about 50 g/min under a product temperature of about 27° C. and an atomization pressure of 1.5 bar, and then dried to a final product temperature of 32° C. The loss on drying (LOD) value was measured using a Computrac™ Max 2000 set at 55° C. and was equal to 0.77%. The resulting granulation was comprised of approximately 50% ibuprofen by weight.

TABLE A
Composition of Ibuprofen Granulation

| | Percent | Batch |
|---|---|---|
| Ingredients | (w/w) | Weight (g) |
| Ibuprofen USP | 50.0 | 3000 |
| Citric Acid USP | 6.7 | 400 |
| Dextrose Monohydrate | 33.7 | 2260 |
| Hypromellose 2910 USP* | 4.1 | 250 |
| Starch NF | 1.5 | 90 |
| TOTAL | 100.0 | 6000 |

*Hypromellose sold under the tradename of Methocel E5

### Part B: Preparation of Taste Masking Coating Solution Formula A

A coating solution was prepared by combining an aqueous dispersion of anionic copolymer of methacrylic acid and methacrylates, which is commercially available from Rohm America, LLC, under the tradename, “Eudragit L 30 D-55,” and an aqueous dispersion of glycerol monostearate (GMS), which is commercially available from Emerson Resources, Inc. under the tradename, “Plasacryl,” in purified water under ambient conditions with mixing via a laboratory mixer at 25 RPM. The resulting dispersion contained 20% solids and was comprised of the ingredients set forth in Table B:

TABLE B

| | Percent |
|---|---|
| Ingredients | (w/w) |
| Eudragit L 30 D-55* (30% Solid dispersion) | 62 |
| PlasACRYL ™** (20% Solid dispersion) | 7 |
| Purified Water | 31 |
| TOTAL | 100.0 |

### Part C: Preparation of Coated Active Ingredient Formula A

Preparation of Coated Ibuprofen Granules: 5000 g of the ibuprofen granulation prepared in accordance with Part A of Example 1 were sequentially coated with the enteric polymer solution prepared in accordance with Part B of Example 1 at a rate of about 55 g/min in a Glatt GPCG-5/9 fluid bed unit with a Wurster insert under product temperature conditions of about 25° C., an air flow of about 4.6 scfm and an atomization air pressure of 2.5 bar. The resulting coated particles contained, based on the weight of the final coated particles, about 9.70% polymer coating and about 40-45% of ibuprofen.

The composition of the dry polymer coating is set forth in Table C:

TABLE C

| | Percent |
|---|---|
| Ingredients | (w/w)* |
| Eudragit L 30 D-55* | 93.0 |
| PlasACRYL ™** | 7.0 |
| TOTAL | 100.0 |

*based upon the total dry weight of the coating:

### Part D: Production of Tablets for Evaluation Thereof Formula A

Preparation of the Tablet Blend Base

TABLE D

| | Percent | |
|---|---|---|
| Ingredients | (w/w) | mg/tab |
| Coated Granulated Ibuprofen* | 15.8 | 221.5 |
| Dextrose Monohydrate Coarse Grade | 77.1 | 1078.9 |
| Crospovidone NF** | 1.7 | 23.4 |
| Orange Flavor | 0.3 | 4.0 |
| Magnesium Stearate NF | 1.6 | 22.4 |
| Colloidal Silicon Dioxide NF | 0.1 | 2.0 |
| Fumaric Acid NF | 0.6 | 8.0 |
| Citric Acid USP | 0.3 | 4.6 |
| FD&C Yellow 6 Aluminum Lake | 0.2 | 3.2 |
| Acesulfame Potassium | 1.1 | 16.0 |
| Sucralose NF | 1.1 | 16.0 |
| TOTAL | 100.0 | 1400.0 |

*45% active, produced in accordance with Part C
**Sold under the tradename Polyplasdone XL10

A 1400.0 g batch using the formula in table D was prepared. All of the materials in Table D above except for the ibuprofen were manually passed through a 30 mesh screen. The resulting mixture along with the coated ibuprofen were then placed into a 4 quart V-Blender and mixed for 5 minutes to yield a tablet base blend.

Preparation of Compressed Tablets:

To prepare the chewable tablet, the tablet base blend was compressed on a rotary tablet press using ⅝-inch troche-shaped round B-type tooling. The tablets were compressed at a weight of 1400 mg with a hardness range of 4˜7 kilopounds.

### Example 2

### Ibuprofen Tablets Formula B

### Part A: Preparation of Ibuprofen Granulation Formula B

A granulation comprised of the ingredients set forth in Table E below was made by initially combining ibuprofen, dextrose, citric acid, and hypromellose with mixing at an air flow rate of 350 scfm in a 90 Liter fluid bed granulator fitted with an 18 inch wurster column and spray gun to form a granulation mixture.

Three batches of a granulating solution were made simultaneously in a 55 gallon stainless steel tank. Using a pneumatic driven mixer fitted with a low shear propeller set at 50 RPM, for each batch of granulation, 480 g of starch was added to 7.99 kg of cold water with mixing to produce a starch paste solution having 5.67% solids. This solution was heated while mixing until it reached 79° C. to yield a granulating solution. This granulating solution was then sprayed into the granulation mixture at about 150-175 g/min under an inlet air temperature of about 46.1° C. and an atomization pressure of 4.14-5.52 bar, then dried using an inlet air temperature of 46.1-54.4° C. for approximately 12.9-22.1 minutes. The loss on drying (LOD) value was measured using a Computrac™ Max 2000 set at 55° C. and was equal to 0.69-0.80%. The resulting granulation was comprised of approximately 50% ibuprofen by weight.

TABLE E
Composition of Ibuprofen Granulation - Formula B

| | Percent | Batch |
|---|---|---|
| Ingredients | (w/w) | Weight (kg) |
| Ibuprofen USP | 50.0 | 16.00 |
| Citric Acid USP | 6.7 | 2.13 |
| Dextrose Monohydrate | 33.7 | 12.05 |
| Hypromellose 2910 USP* | 4.1 | 1.33 |
| Starch NF | 1.5 | 0.48 |
| TOTAL | 100.0 | 32.0 |

*Sold under the tradename Methocel E5

### Part B: Preparation of Taste Masking Coating Solution Formula B

A coating solution was prepared by combining an aqueous dispersion of anionic copolymer of methacrylic acid and methacrylates, which is commercially available from Rohm America, LLC, under the tradename, “Eudragit L 30 D-55,” and an aqueous dispersion of glycerol monostearate (GMS), which is commercially available from Emerson Resources, Inc. under the tradename, “Plasacryl,” in purified water under ambient conditions with mixing via a mixer at 25 RPM. The resulting dispersion contained about 20% solids and was comprised of the ingredients set forth in Table F:

TABLE F
Composition of Coating Solution - Formula B

| | Percent |
|---|---|
| Ingredients | (w/w) |
| Eudragit L 30 D-55* (30% Solid dispersion) | 64 |
| PlasACRYL ™** (20% Solid dispersion) | 4 |
| Purified Water | 32 |
| TOTAL | 100.0 |

### Part C: Preparation of Coated Active Ingredient Formula B

Preparation of Coated Ibuprofen Granulation: 27.95 kg of the ibuprofen granulation prepared in accordance with Part A of Example 2 were sequentially coated with the enteric polymer solution prepared in accordance with Part B of Example 2 at a rate of about 220 g/min in a 90 Liter fluid bed unit with an 18 inch wurster insert under product temperature conditions of about 50.0-58.3° C., an air flow of about 440-610 scfm and an atomization air pressure of 3.45-4.14 bar. The particles were then dried using an inlet temperature of about 50.0-57.8° C. for 3.6 to 19.9 minutes. The coated particles contained, based on the weight of the final coated particles, about 15.79% polymer coating and about 40-45% of ibuprofen.

The composition of the dry coating is set forth in Table G:

TABLE G

| | Percent |
|---|---|
| Ingredients | (w/w)* |
| Eudragit L 30 D-55* | 93.0 |
| PlasACRYL ™** | 7.0 |
| TOTAL | 100.0 |

*based upon the total dry weight of the coating:

### Part D: Production of Tablets for Evaluation Thereof—Formula B

Preparation of the Tablet Blend Base

TABLE H
Tablet Base Blend Formula B

| | Percent | |
|---|---|---|
| Ingredients | (w/w) | mg/tab |
| Coated Granulated Ibuprofen* | 17.3 | 241.5 |
| Dextrose Monohydrate (Coarse Grade) | 73.8 | 1032.6 |
| Crospovidone NF** | 2.1 | 29.0 |
| Art Grape Flavor | 0.3 | 4.5 |
| Magnesium Stearate NF | 1.07 | 15.0 |
| Colloidal Silicon Dioxide NF | 0.14 | 2.0 |
| Fumaric Acid NF | 3.29 | 46.0 |
| Citric Acid USP | 0.21 | 3.0 |
| FD&C Blue L#1 Aluminum Lake | 0.10 | 1.4 |
| D&C Red Calcium Lake | 0.07 | 1.0 |
| Acesulfame Potassium | 1.07 | 15.0 |
| Sucralose NF | 0.64 | 9.0 |
| TOTAL | 100 | 1400.0 |

*41.4% active, produced in accordance with Part C
**Sold under the tradename Polyplasdone XL-10

A 1400 g batch using the formulation in Table H was prepared. The citric acid, sucralose, colloidal silicon dioxide, fumaric acid, crospovidone (Polyplasdone XL-10), and flavor were placed into a plastic bag to form a sucralose mixture.

The Coated Granulated Ibuprofen particles produced in Part C of Example 2 were placed into a 2 quart V-Blender. The Acesulfame potassium was passed through a 14 mesh screen, then added to the blender. The colors were passed through a 30 mesh screen, then added to the blender. The dextrose monohydrate was passed through a 14 mesh screen, then added to the blender. The sucralose mixture was then screened through a 30 mesh screen, added to the blender, and the resulting mixture was blended for 3 minutes. The magnesium stearate was screened through a 30 mesh screen, then added to the blender and blended for 3 minutes to yield a tablet base blend.

Preparation of Compressed Tablets:

To prepare the chewable tablet, the tablet base blend was then compressed on a rotary tablet press using ⅝-inch troche-shaped round B-type tooling. The tablets were compressed at a weight of 1400 mg with a hardness range of 4˜7 kilopounds.

### Example 3

### Ibuprofen Tablets Formula C

### Part A: Production of Tablets for Evaluation Thereof—Formula C

Preparation of the Tablet Blend Base

TABLE I
Tablet Base Blend Formula C

| | Percent | |
|---|---|---|
| Ingredients | (w/w) | mg/tab |
| Coated Granulated Ibuprofen* | 17.3 | 241.5 |
| Dextrose Monohydrate (Coarse Grade) | 75.4 | 1055.6 |
| Crospovidone NF** | 2.1 | 29.0 |
| Art Grape Flavor | 0.3 | 4.5 |
| Magnesium Stearate NF | 1.07 | 15.0 |
| Colloidal Silicon Dioxide NF | 0.14 | 2.0 |
| Fumaric Acid NF | 1.64 | 23.0 |
| Citric Acid USP | 0.21 | 3.0 |
| FD&C Blue L#1 Aluminum Lake | 0.10 | 1.4 |
| D&C Red Calcium Lake | 0.07 | 1.0 |
| Acesulfame Potassium | 1.07 | 15.0 |
| Sucralose NF | 0.64 | 9.0 |
| TOTAL | 100.0 | 1400.0 |

*41.4% active, produced in accordance with Parts A-C, Example 2.
**Sold under the tradename Polyplasdone XL10

A 1400 g batch using the formulation in Table I was prepared. The citric acid, sucralose, colloidal silicon dioxide, fumaric acid, crospovidone (Polyplasdone XL-10), and flavor were placed into a plastic bag to form a sucralose mixture.

The Coated Granulated Ibuprofen particles produced in Part C of Example 2 were placed into a 2 quart V-Blender. The Acesulfame potassium was passed through a 14 mesh screen, then added to the blender. The colors were passed through a 30 mesh screen, then added to the blender. The dextrose monohydrate was passed through a 14 mesh screen, then added to the blender. The sucralose mixture was passed through a 30 mesh screen and then added to the blender, and the resulting mixture was blended for 3 minutes. The magnesium stearate was screened through a 30 mesh screen, then added to the blender and blended for 3 minutes to yield a tablet base blend.

Preparation of Compressed Tablets:

To prepare the chewable tablet, the tablet base blend was then compressed on a rotary tablet press using ⅝-inch troche-shaped round B-type tooling. The tablets were compressed at a weight of 1400 mg with a hardness range of 4˜7 kilopounds.

### Example 4

### Ibuprofen Tablets Formula D

### Part A: Production of Tablets for Evaluation Thereof—Formula D

Preparation of the Tablet Blend Base

TABLE J
Tablet Base Blend Formula D

| | Percent | |
|---|---|---|
| Ingredients | (w/w) | mg/tab |
| Coated Granulated Ibuprofen* | 17.2 | 241.6 |
| Dextrose Monohydrate (Coarse Grade) | 76.6 | 1072.9 |
| Crospovidone NF** | 2.1 | 29.0 |
| Art Mango Flavor | 0.5 | 7.0 |
| Magnesium Stearate NF | 1.07 | 15.0 |
| Colloidal Silicon Dioxide NF | 0.14 | 2.0 |
| Fumaric Acid NF | 1.57 | 22.0 |
| Citric Acid USP | 0.21 | 3.0 |
| Acesulfame Potassium | 0.43 | 6.0 |
| Sucralose NF | 0.18 | 2.5 |
| TOTAL | 100.0 | 1401 |

*41.4% active, produced in accordance with Parts A-C, Example 2.
**Sold under the tradename Polyplasdone XL10.

A 1400 g g batch using the formulation in Table J was prepared. The citric acid, sucralose, colloidal silicon dioxide, fumaric acid, crospovidone (Polyplasdone XL-10), and flavor were combined in a plastic bag. These materials were passed through a 30 mesh screen. The Coated Granulated ibuprofen was then added to the bag and blended. The dextrose monohydrate was passed through a 14 mesh screen, then added to the bag and blended. The magnesium stearate was passed through a 30 mesh screen, then added to the bag and blended. The sweeteners were then added to the bag and blended to yield a tablet base blend.

Preparation of Compressed Tablets:

To prepare the chewable tablet, the tablet base blend was then compressed on a rotary tablet press using ⅝-inch troche-shaped round B-type tooling. The tablets were compressed at a weight of 1401 mg with a hardness range of 4˜7 kilopounds.

### Example 5

### Analysis of Dissolution Data

The tablets produced in the above Examples 2, 3 and 4 were analyzed using the following dissolution analysis: USP Type II apparatus (paddles, 50 RPM) in pH 5.6 acetate buffer at 37° C. for 60 minutes. Dissolution samples were analyzed for ibuprofen content versus a standard prepared at the theoretical concentration for each timepoint using an Agilent® UV spectrophotometer set at a wavelength of 220 nm using a 1 cm flow-cell.

The ibuprofen tablets made in accordance with Example 2, 3, and 4 possessed a 100% release in the pH 5.6 buffer at 60 minutes.

### Example 6

### Evaluation of Throat Burn

Samples of the tablets produced in Example 2, 3, and 4, and a commercially-available chewable ibuprofen tablet available from McNEIL Consumer Healthcare under the tradename, “Motrin® Junior Strength chewable tablet,” were evaluated by a panel of 20 panelists in a blinded study for i) throat burn and numbness; and ii) tongue/cheek/mouth burn during mastication. Panelists in this study were pre-screened as being sensitive to the burning sensation of ibuprofen. Using a monadic design, the panelists were instructed to chew and swallow one tablet, then wait 2-4 minutes before evaluating. They were instructed to rate the burn of the tablets as follows: 1=no burn; 2=slight burn; 3=moderate burn; 4=high burn; and 5=very high burn. They rated three hedonic attributes including overall liking, taste/flavor, and aftertaste and two intensity attributes including throat burn/numbness and tongue/cheek/mouth burn. This procedure was repeated about every two days with each panelist, but with the replacement of the evaluated tablet with another one of the remaining, unevaluated tablets mentioned above.

As shown in Table K, this Example showed that the tablets produced in accordance with the present invention possessed significantly lower throat burn/numbness and tongue/cheek/mouth burn relative to that possessed by the commercial product.

TABLE K
Taste Comparison Data

| | Commercial | Tablets - | Tablets - | Tablets - |
|---|---|---|---|---|
| Attribute | tablet | Example 2 | Example 3 | Example 4 |
| Throat | 3.6 (0.7) | 2.4 (0.8) | 2.7 (1.0) | 3.0 (1.2) |
| Burn/Numbness | | | | |
| Tongue/Cheek/Mouth | 3.6 (0.9) | 2.5 (1.1) | 2.4 (1.1) | 2.7 (0.8) |
| Burn | | | | |

Mean (Standard Deviation)

We claim:

1. An immediate release dosage form capable of being chewed or disintegrated in the oral cavity prior to swallowing, comprising:
a. a plurality of particles comprising
(i) ibuprofen and/or pharmaceutically acceptable salts thereof, and
(ii) a taste-masking effective amount of a first water soluble acid having a solubility greater than about 10 g/100 mL water at 20° C.; and
b. a matrix comprising a second water soluble acid having a solubility less than about 5 g/100 mL water at 20° C.

2. The dosage form of claim 1, wherein the first water soluble acid is selected from the group consisting of alanine, arginine, glucine, proline, lycine, threonine glutaric acid, ascorbic acid, malic acid, oxalic acid, malonic acid, acetic acid, tartaric acid, citric acid, and mixtures thereof.

3. The dosage form of claim 1, wherein the second water soluble acid is selected from the group consisting of oleic acid, stearic acid, aspartic acid, glutamic acid, glutamine, histidine, isoleucine, leucine, methionone, phenylalanine, serine, tryptophan, tyrosine, valine, fumaric acid, and mixtures thereof.

4. The dosage form of claim 1, wherein the weight ratio of the first water soluble acid to the second water soluble acid is from about 1 part to about 50 parts:about 99 parts to about 50 parts.

5. The dosage form of claim 1, wherein the weight ratio of the first water soluble acid to the second water soluble acid is from about 1 part to about 10 parts:about 99 parts to about 90 parts.

6. The dosage form of claim 1, wherein the dosage form is comprised of, based upon the total weight of the dosage form, from about 1 percent to about 50 percent of the particles and from about 50 percent to about 99 percent of the matrix.

7. The dosage form of claim 1, wherein the dosage form is comprised of, based upon the total dry weight of the coated particles, from about 25 percent to about 80 percent of ibuprofen and from about 5 percent to about 15 percent of the first water soluble acid.

8. The dosage form of claim 1, wherein the dosage form is comprised of, based upon the total weight of the dosage form, from about 0.1 percent to about 20 percent of the second water soluble acid.

9. The dosage form of claim 1, wherein the particles, the matrix, or both the particles and the matrix are further comprised of a secondary active ingredient selected from the group consisting of bisacodyl, famotidine, ranitidine, cimetidine, prucalopride, diphenoxylate, loperamide, lactase, mesalamine, bismuth, antacids, and pharmaceutically acceptable salts, esters, isomers, and mixtures thereof.

10. The dosage form of claim 1, wherein the particles, the matrix, or both the particles and the matrix are further comprised of a secondary active ingredient selected from the group consisting of acetaminophen, acetyl salicylic acid, naproxen, ketoprofen, flurbiprofen, diclofenac, cyclobenzaprine, meloxicam, rofecoxib, celecoxib, and pharmaceutically acceptable salts, esters, isomers, and mixtures thereof.

11. The dosage form of claim 1, wherein the particles, the matrix, or both the particles and the matrix are further comprised of a secondary active ingredient selected from the group consisting of pseudoephedrine, phenylpropanolamine, chlorpheniramine, dextromethorphan, diphenhydramine, astemizole, terfenadine, fexofenadine, loratadine, cetirizine, mixtures thereof and pharmaceutically acceptable salts, esters, isomers, and mixtures thereof.

12. The dosage form of claim 1, wherein the dosage form is in the form of a tablet.

13. The dosage form of claim 1, wherein the particles are coated with a polymeric coating layer.

14. The dosage form of claim 13, wherein the polymeric coating layer is comprised of an enteric polymer.

15. An immediate release dosage form capable of being chewed or disintegrated in the oral cavity prior to swallowing, comprising, based upon the total weight of the dosage form:
a. from about 1 percent to about 50 percent of a plurality of particles, said particles comprising, based upon the total weight of the particles,
(i) from about 25 percent to about 80 percent of ibuprofen, and
(ii) from about 1 percent to about 20 percent of a first water soluble acid having a solubility greater than about 10 g/100 mL water; and
b. from about 50 percent to about 99 percent of a matrix comprising, based upon the total weight of the dosage form, from about 0.1 percent to about 20 percent of a second water soluble acid having a solubility less than about 5 g/100 mL water.

16. The dosage form of claim 15, wherein the particles are coated with a taste-masking polymeric coating layer.

17. The dosage form of claim 16, wherein the polymeric coating layer is comprised of an enteric polymer.
